(19) United States
(12) Patent Application Publication
(10) Pub. No.: US 2022/0023274 A1
(43) Pub. Date: Jan. 27, 2022

(54) USE OF A BERBERIS AND RESVERATROL MIXTURE TO CONTROL DYSLIPIDEMIA

(71) Applicant: S&R FARMACEUTICI S.P.A., Bastia Umbra (IT)

(72) Inventors: Bernard FIORETTI, Foligno (IT); Lucio LEONARDI, Gualdo Cattaneo (IT)

(21) Appl. No.: 17/311,669

(22) PCT Filed: Dec. 16, 2019
(86) PCT No.: PCT/IB2019/060873

§ 371 Date: Jun. 7, 2021

(30) Foreign Application Priority Data
Dec. 17, 2018 (IT) 102018000011155

(51) Int. Cl. A61K 31/4375; A61K 31/05; A23L 33/10; A61K 45/06; A61P 3/06

(52) U.S. Cl. CPC A61K 31/4375; A61K 31/05; A61P 3/06; A61K 45/06; A23L 33/10

(57) ABSTRACT

A pharmaceutical or nutraceutical composition or a food supplement including a mixture of berberine and resveratrol for use in treatment and/or control of dyslipidemia is described, wherein resveratrol is co-precipitated or supported on a salt or hydroxide of a divalent or trivalent metal.

### APPLICATION FIELD

This invention regards a pharmaceutical, nutraceutical or dietary supplement composition containing a mixture of berberis and resveratrol for use in the control of dyslipidemia.

### PRIOR ART

Dyslipidemia is the variation in the amount of lipids circulating in the blood, especially cholesterol, triglycerides, phospholipids, much more frequently increasing (hypercholesterolemia, hypertriglyceridemia, hyperfosfolipidemia), more rarely decreasing. The increase in plasma cholesterol, triglycerides, or of both, or low-density lipoproteins (LDL) contribute to the development of atherosclerosis and cardiovascular risk.

The range of interventions that can be used to control LDL cholesterol focuses on diet correction, the use of drugs, mainly statins, and nutraceutical agents administered as dietary supplements or functional foods. An adequate supply of fiber, equipped with both metabolic and prebiotic effects, of phytocompounds (polyphenol species) with antioxidant, anti-inflammatory and prebiotic action, polyunsaturated fatty acids with anti-inflammatory, anti-aggregating effects, anti-arrhythmia, potassium, antipertensive action, characterizes a balanced nutritional pattern, rich and varied, which can contribute widely and significantly to the reduction of cardiovascular risk and mortality associated with dyslipidemia.

Statins, are a first-line drug in the management of lipid disorders, are burdened, in a not negligible percentage of cases, by side effects, such as myalgia (about 10%), or more rarely hepatotoxicity.

In patients who follow only dietary treatment strategies, goals can be achieved better and are more stable. There are several molecules with a potential lipid reducing action and therefore usable in the prevention of dyslipidemia and therefore cardiovascular risk.

Resveratrol (3,5,4′-trihydroxystilbene) is a polyphenol with a stilbenic structure contained in various foods such as wine and is particularly enriched in 5 phytocomplexes such as those attracted by the Polygonum cuspidatum rhizome. Resveratrol shows anti-oxidant, anti-inflammatory anti-viral, cardio-protective, neuro-protective, anti-cancer and anti-angiogenetic activities. Recently it has been observed in obese human subjects that treatment with trans-resveratrol reduces glucose levels, triglycerides and inflammation with an effect similar to that induced by caloric restriction. However, according to a recent meta-analysis, resveratrol does not show hypophasmic effects (total cholesterol, LDL and HDL) (Sahebkar A. Effects of resveratrol supplementation on plasma lipids: a systematic review and meta-analysis of randomized controlled trials. Nutr Rev. 2013 December; 71(12):822-35).

Resveratrol is a molecule that due to its low water solubility has a reduced absorption that is reflected in its limited bioavailability. Absorption of resveratrol is further diminished by the presence of a biological instability that converts the molecule into it its metabolites such as sulfates and glucoronates.

Berberine is an alkaloid extracted from the root of plants of the genus Berberis (B. Aristata and other spp). The berberine receptor that averages the biological effects has not been yet identified with certainty, but it is believed that the main mechanism by which this molecule acts upon lipid metabolism is through the increase in the LDL cholesterol liver clearance. Berberin has proven effective in controlling the overall lipid profile (including plasma levels of triglycerides and HDL cholesterol) and blood sugar. Berberine possesses various mechanisms currently being studied.

On the one hand, it seems able to reduce the levels of messenger RNA (mRNA) for PCSK9 (Proprotein Convertasi Subtilisine/Kexina type 9), and therefore the plasma levels of this protein. PCSK9, produced primarily by the liver, binds, as it is known to LDL receptors, decreasing its ability to return to the cell surface after completing the cycle of internalization and degradation of an LDL. PCSK9, therefore, ultimately reduces the presence of LDL receptors on the surface of hepatocytes. Berberin, on the other hand, would also have more specific gene effects, stabilizing the mRNA that encodes for the LDL receptor (LDLR).

The combination of these two mechanisms (mRNA stabilization and reduced PCSK9 activity) would lead to an increased presence of the receptor on the surface of the hepatocyte and consequently increases cell capture of LDLs, reducing their plasma levels. Triglycerides are also reduced as a result of Berberin, probably due to modulation of the MAP-Kase activity (inhibition) and AMP-Kase (stimulation). HDL cholesterol levels would increase by a few percentage points. Berberine has a high level of tolerance within the daily dose currently used (500-1500 mg/die). For safety reasons the recommended daily dose to maintain control of cholesterol and plasma triglycerides is 500 mg.

In the patent application number CN1539419, it is reported that resveratrol inhibits the accumulation of cholesterol and triglycerides in the liver, increases HDL levels, regulates the ratio of lipoproteins and prevents the oxidation of LDLs, all actions within the lipid metabolism. Berberine, similarly reduces levels of total cholesterol and triglycerides. The above application states that the combination of the two actives, resveratrol and berberine, can regulate blood lipids better than resveratrol and Berberin taken individually, but no experimental supporting evidence is reported. Differently, evidence of an effect on HIV antiviral action and senile dementia is reported. The same application describes examples of oral capsules, tablets and injection solutions.

### SUMMARY OF INVENTION

The applicant has now surprisingly found that the property of reducing the concentration of Lipoprotein LDL by berberine is increased when it is associated with a resveratrol that has a gastric uptake compared to intestinal one.

Therefore, in accordance with the invention, a pharmaceutical, nutraceutical or in the form of a dietary supplement composition is disclosed, the composition containing a mixture of berberine and resveratrol for use in the control of dyslipidemia, wherein resveratrol is contained in a formulation in which it is co-precipitated or supported on salts or hydroxide of bivalent or trivalent metals.

The absorption of resveratrol is conditioned by the time of dissolution and the time of gastric emptying. In general the absorption of resveratrol begins at the gastric level, but the main site of absorption is the intestinal one. The use of formulations such as those in the EP 2679243 patent, the content of which is fully incorporated here for reference, allow to increase the absorption of the active component at the gastric level. In fact, the plasma profile of these resveratrol formulations are characterized by a shorter short peak time than that associated with pure resveratrol. The resveratrol molecule has an optimal distribution coefficient to cross the biological membranes, but the absorption and therefore its bioavailability is limited by poor water solubility.

Molecules as resveratrol (permeable to membranes, but poorly soluble in water) are classified according to the biopharmaceutical system in the class II, and their absorption can be improved by increasing the rate of dissolution. Formulations of resveratrol co-precipitated or supported with bivalent or trivalent metal salts, known in art as described in the patent application EP 2679243 have an improved dissolution rate compared to a pure resveratrol that is not supported. According to a particularly preferred form of realization, a formulation containing trans-resveratrol supported on magnesium hydroxide is used in the composition according to the invention. The content of trans-resveratrol in this formulation can be between 10% and 50% in weight on the weight of the formulation, preferably it is 30% weighted on the weight of the formulation and sold commercially under the trade name REVIFAST®.

REVIFAST® has an increased dissolving rate at the pH in the gastric environment compared to unsupported pure resveratrol. Differently, berberine is poorly bioavailable (Liu et al., 2016) and its absorption is at the intestinal level by a mechanism not yet known, but probably linked to the reductant action of the intestinal flora (Liu C S, Zheng Y R, Zhang Y F, Long X Y. Research progress on berberine with a special focus on its oral bioavailability. Phytotherapy. 2016 March; 109:274-82; Feng R, Shou J W, Zhao Z X, He C Y, Ma C, Huang M, Fu J, Tan X S, Li X Y, Wen B Y, Chen X, Yang X Y, Ren G, Lin 20 Y, Chen Y, You X F, Wang Y, Jiang J D. Transforming berberine into its intestine-absorbable form by the gut microbiota. Ski Rep. 2015 Jul. 15; 5:12155).

The present invention will be explained in more details below also with reference with the attached drawings wherein:

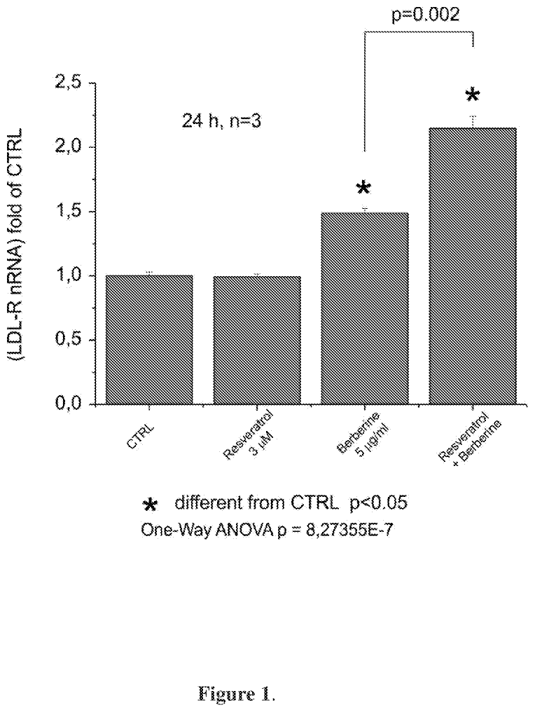

FIG. 1 shows the effects on the transcriptional expression of the Low-Density lipoprotein Receptor (LDL-R) compared to the CTRL condition (no treatments) of Resveratrol (3 μM, 24 hours), Berberine (5 μg/mL, 24 hours of treatment) and both co-application (Resveratrol 3 μM+Berberine5 μg/mL, 24 hours); and

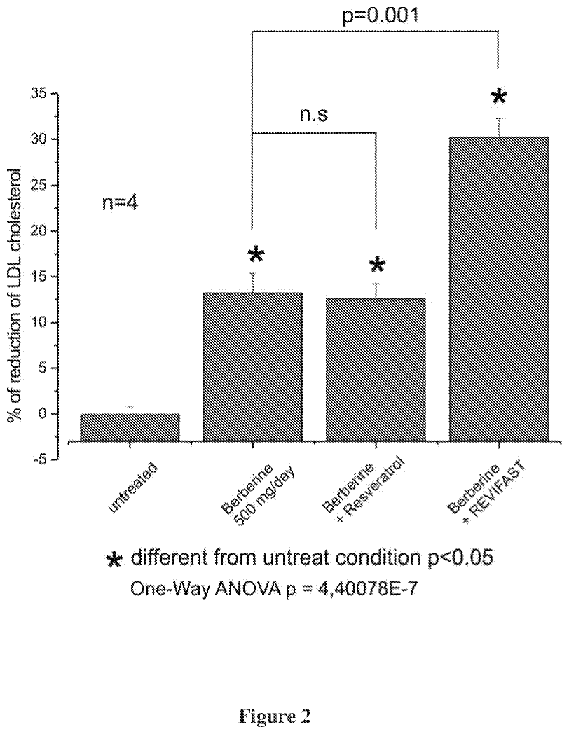

FIG. 2 shows the effect on plasma LDL concentration as percentage reduction compared to nontreated condition, of berberine (500 mg) alone or with 48 mg of resveratrol (also supported on magnesium hydroxide, REVIFAST) during feeding scheme similar to than described in example 9 (n=4). Note the absence of synergism in LDL reduction with a combination of berberine with 48 mg pure resveratrol. In contrast synergic effects was observed with combination of berberine with 48 mg of resveratrol supported on magnesium hydroxide (160 mg of REVIFAST). The four subjects had a comparable initial plasma LDL concentration.

### DETAILED DESCRIPTION

In a preferred form of realization, this invention concerns a pharmaceutical or nutraceutical composition comprising a resveratrol supported on bivalent or trivalent metal hydroxide and berberine in specific stoichiometric ratios for use in the control of dyslipidemia. In particular, such a composition is able to:

a. Synergistically increase the liver expression of LDLRs;

b. improve the overall lipid profile and thus reduce cardiovascular risk.

As known in the prior art, the supplementation in the diet of resveratrol does not result in a change of the levels of LDL whereas the levels of LDL are clearly reduced by the supplementation of berberine alone. In addition, the supplementation in the diet of a composition containing unsupported resveratrol and berberine only provides a slight reduction of the LDL levels compared to supplementation of berberine alone (see particularly example 9).

However, in accordance with the present invention, the supplementation in the diet of a composition containing berberine and resveratrol co-precipitated or supported on salts or hydroxide of bivalent or trivalent metals, such as Revifast®, results surprisingly in a reduction of the LDL levels that is considerable higher than that expected by the sum of the effects of resveratrol and berberine alone or by the effect of the combination of berberine and unsupported resveratrol. Therefore, the combination of berberine and supported resveratrol clearly shows synergy in further reducing LDL cholesterol (see particularly example 9).

Without wishing to be bound to any scientific theory, an explanation for the above results can be found in that it is the way in which resveratrol is absorbed that influences the synergistic action of resveratrol to reduce LDL cholesterol by berberine.

The composition according to the invention may include in addition to berberine and resveratrol, the latter preferably supported on magnesium hydroxide, nutrients chosen by the group consisting of Astragalus microcephalus, Fermented Red Rice, garlic, catechins from the green tea, phytocomplexes derived from Acacia catechu, Acacia farnesiana, Acacia laeta, Acacia vera, Acacia senegal, Acacia seyal delile, Amaranthus caudatus, Amaranthus cruentus, Amaranthus hypocondria, Cassia mimosoides var. Nomame makino, Cyclantera pedata Schard (Caigua), Hypomea batatas Lamb., Trigonella foenumgraecum, β-glucan polysaccharides and their combinations.

Astragalus microcephalus polysaccharides, obtained from the plant's root extract are the main effective molecules. Previous studies have shown some effectiveness in controlling cholesterol levels through the reduction of cholesterol absorption. The most recently described mechanism of action shows that the hypocholesterolemizing action of astralagalus polysaccharides is likely due to multi-target action. Astragalus m. lowers plasmatic cholesterol through a combination of actions; inhibition of its absorption, increased excretion through fecal bile acid, up-regulation of the liver LDL-R receptor and the expression of the cyp7a-1a gene which has effects on fecal excretion or absorption (Cheng Y, Tang K, Wu S, Liu L, Qiang C, Lin X, Liu B. Astragalus polysaccharides lowers plasma cholesterol through distinct mechanisms distinct from statins PLoS One. 2011; 6(11):e27437).

Various products and active ingredients have also proven useful in controlling dyslipidemias such as fermented red rice, garlic and green tea catechins (Hunter P M, Hegele R A. Functional foods and dietary supplements for the management of dyslipidemia. Nat Rev Endocrinol. 2017 May; 13(5):278-288).

Fermented Red Rice is a complex formed by the fermentation of rice by Monascus purpureus widely used in traditional Chinese medicine and also in the West. The active ingredient to which the hypoforeigning action is ascribed is the monacolin K, a natural statin and inhibitor of hydroxy-methyl Coenzyme A reductase (HMCoA-reductase), biochemical step limiting cholesterol biosynthesis. Typically, the concentration in monacolin k found in commercial extracts ranges from 1 to 10% and in some legislations the amount of monacolin K administered with supplements is regulated. For example, in Italy this daily limit is set at 10 mg. Fermented red rice is widely used for cholesterol control, despite some cases of manifestations of side effects similar to those of statins.

Garlic reduces cholesterol levels by inhibiting HMCoA-reductase and sterol alfa methyl oxidase activity. These actions would appear to be due to the action of the active ingredient Allicin. To have a cholesterol reduction of about 5% the recommended daily dose of allicin is 10 mg.

The presence of catechins, a class of flavonoids, is thought to be responsible for the cholesterol-lowering effects of green tea. The recommended daily dose to have effects on lipidemia must be greater than 200 mg.

β-glucan polysaccharides are components of mycetes' cell walls (examples from spp. Pleurotus) that may participate in physiological processes related to the metabolism of fats in the human body. Their application results in a decrease in the total cholesterol contentin blood and may also contribute to reductions in body weight (Rop O, Mlcek J, Jurikova T. Beta-glucans in higher fungi and their health effects. Nutr Rev. 2009 November; 67(11):624-31).

Other phytocomplexes shown regulating cholesterol metabolism are: Acacia catechu, Acacia farnesiana, Acacia laeta, Acacia vera, Acacia senegal, Acacia seyal delile, Amaranthus caudatus, Amaranthus cruentus, Amaranthus hypocondria, Cassia mimosoides var. Nomame makino, Cyclantera pedata Schard (Caigua), Hypomea batatas Lamb., Trigonella foenumgraecum. (See for example Kabiri N, Asgary S, Setorki M. Lipid lowering by hydro extracts of Amaranthus caudatus L. induces regression of rabbits atherosclerotic lesions. Lipids Health Dis. 2011 May 5 28; 10:89; Attia E S, Amer A H, Hasanein M A. The hypoglycemic and antioxidant activities of garden cress (Lepidium sativum L.) seed on alloxan-induced diabetic bad rats. Nat Prod Res. 2017 December 13:1-5; Knott E J, Richard A J, Mynatt R L, Ribnicky D, Stephens J M, Bruce-Keller A. Fenugreek supplementation during high-fat feeding improves specific markers of metabolic health. Ski Rep. 2017 Oct. 6; 7(1):12770; Effects of potato and lotus leaf extract intake on body composition and blood lipid concentration. J Exerc Nutrition Biochem. 2015 March; 19(1):25-30 and Ried K, Toben C, Fakler P. Effect of garlic on serum lipids: an updated meta-analysis. Nutr Rev. 2013 May; 71(5):282-99).

Preferably, in composition according to the invention, the amount of total gastric absorption resveratrol administered daily is between 20 mg and 200 mg, preferably between 30 and 150 mg even more preferably 50 mg. Preferably, in composition according to invention, gastric absorption resveratrol is a resveratrol supported on bivalent or trivalent metal hydroxy (particularly resveratrol derived from REVIFAST®).

Preferably, in composition according to the invention, the total amount of berberine administered daily is between 20 mg and 2000 mg, preferably between 250 and 1500 mg even more preferably 500 mg.

Preferably, Berberin is obtained from a Berberis aristata extract with a Berberine title between 50 and 100%, preferably between 70 and 90% even more than preferably 85%.

In composition according to the invention, the weight ratio between resveratrol and berberin preferably ranges from 0.01 to 1, preferably between 0.05 to 0.5 even more preferably 0.1.

Preferably, in composition according to the invention, the amount of 5 Astragalus microcephalus is between 10 mg and 500 mg, preferably between 15 and 100 mg even more preferably 20 mg on a total weight of composition comprised between 0.5 and 2 grams, preferably between 0.75 and 1.5 grams even more preferably 1 gram.

Preferably, in the composition according to the invention, the amount of fermented red rice is between 50 mg and 500 mg, preferably between 100 and 300 mg even more preferably 200 mg on a total weight of composition between 0.5 and 2 grams, preferably between 0.75 and 1.5 grams even more preferably 1 gram.

Preferably, in the composition according to the invention, the amount of monocolin k is between 1 mg and 20 mg, preferably between 2 and 10 mg even more preferably 3 mg on a total weight of composition between 0.5 and 2 grams, preferably between 0.75 and 1.5 even more preferably 1 gram.

Preferably, in the composition according to the invention, the amount of allicin is between 1 mg and 100 mg, preferably between 3 and 30 mg even more 20 preferably 10 mg on a total weight of composition between 0.5 and 2 grams, preferably between 0.75 and 1.5 even more preferably 1 gram.

Preferably, in the composition according to the invention, the amount of catechins is between 50 mg and 500. mg, preferably between 100 and 300 mg even more preferably 200 mg out of a total weight of composition between 0.5 and 2 grams, preferably between 0.75 and 1.5 grams even more preferably 1 gram.

Preferably, in the composition according to the invention, the amount of β-glucan polysaccharides is between 50 mg and 10 grams, preferably between 500 mg and 5 grams mg even more preferably 3 grams on a total weight of composition between 0.5 and 10 grams, preferably between 0.75 and 5 grams even more preferably 3 grams.

Preferably, in the composition according to the invention, the amount of derivatives of phytocomplexes from Acacia catechu, Acacia farnesiana, Acacia laeta, Acacia vera, Acacia senegal, Acacia seyal delile, Amaranthus caudatus, Amaranthus cruentus, Amaranthus cassia mimosoides var. Nomame makino, Cyclantera pedata Schard (Caigua), Hypomea batatas Lamb., Trigonella foenumgraecum is between 0 mg and 500 mg, preferably between 5 and 300 mg even more preferably 10 mg on a total weight of composition between 0.5 and 2 grams, preferably between 0.75 and 1.5 grams even more preferably 1 gram.

The composition according to the invention can be made/produced in a variety of formulations such as: capsules, tablets, syrups, suspensions, stick pack and emulsions and can be supplemented with nutrients that are known for the regularization of the lipid framework.

In addition, such formulations may include any excipients, technological additives, co-forms, polar and semipolar polymer matrices, carriers and/or stabilizers for both pharmaceutical and nutraceutical use.

Examples of excipients are xanthan gum and guar gum, sweeteners such as glucose and sucrose, acidifying as citric acid and sliding agents such as stearic acid. Compositions according to the invention can be administered by splitting them several times a day, preferably once a day.

### EXAMPLES

This invention will now be described with reference to the following examples provided as indicative and not limiting to such.

### Example 1. Effects of the Resveratrol/Berberine Mix in Vitro

Berberine at the concentration of 5 g/ml applied alone tends to increase the transcription of the LDL receptor in hepatocytes in vitro. Differently, resveratrol 3, M applied alone shows no apparent effect on the transcription of the gene under consideration. Surprisingly when applied together there is a significant increase in the expression of the gene greater than the sum of the two active. The increase to about 50% indicating that berberine when applied with resveratrol is more active on its target. It could be said that the concentration tested is bio-equivalent to that of Berberine with a concentration of 50% higher than that administered according to the work of Kong et to 2004. (Kong W, Wei J, Abidi P, Lin M, Inaba S, Li C, Wang Y, Wang Z, Si S, Pan H, 10 Wang S, Wu J, Wang Y, Li Z, Liu J, Jiang J D. Berberine is a novel cholesterol-lowering drug working through a unique mechanism distinct from statins. Nat Med. 2004 December; 10(12):1344-51)).

In agreement with the above, we have found that the levels of LDL receptor mRNA (LDL-R, central to the cholesterol clearance associated with LDL lipoprotein) are not regulated by resveratrol, but are increased by berberine that is known to do so through this mechanism to reduce LDL cholesterol. Surprisingly, when the two substances are combined together, the expression of the receptor increases by 50% compared to berberine alone. Resveratrol being without effect alone is therefore a synergy, never observed before. The innovation is therefore in having observed a synergy between resveratrol and berberine with resveratrol which enhances the effects of berberine in the expression of LDL-R. The results are summarized in FIG. 1.

### Example 2

An exemplary formulation of the composition according to Invention is described below:

| Component | Dose |
|---|---|
| REVIFAST ® | 160 mg (of which 48 mg of |
| 30% weight resveratrol | resveratrol) |
| Berberine | 500 mg |

The above composition can be contained in a variety of formulations such as: capsules, tablets, syrups, suspensions and emulsions. The formulation of example 2 can be supplemented with nutrients that are known for the regularization of the lipid profile.

### Example 3

An exemplary formulation of the composition according to Invention is described below

| Component | Dose | |
|---|---|---|
| REVIFAST ® | 160 mg (of which 48 mg of | |
| 30% weight resveratrol | resveratrol) | |
| Berberine | 500 | mg |
| Astragalus microcephalus | 20 | mg |
| Willd, root extract tit at 70% | | |
| in Polysaccharides | | |

The composition described in Example 3 can be contained in a variety of formulations such as: capsules, tablets, syrups, suspensions and emulsions. The formulations of Examples 2 and 3 also include possible excipients, technological additives, co-formulations, polar and semipolar polymeric matrices, carriers and stabilizers both for pharmaceutical and nutraceutical use. Examples of excipients are xanthan gum and guar gum, sweeteners such as glucose and sucrose, acidifiers such as citric acid and flow agents such as stearic acid. The formulations of examples 2 and 3 can be administered by splitting them several times a day, preferably twice a day.

### Example 4

An exemplary formulation of the composition according to Invention is described below

| Component | Dose | |
|---|---|---|
| Standardized extract of 85% | 588 mg | |
| bark of Berberis aristata DC | | |
| extract in berberine hydrochloride | | |
| REVIFAST ® | 160 mg (of which 48 mg of | |
| 30% weight resveratrol | resveratrol) | |
| 70% magnesium dihydroxide | | |
| Astragalus microcephalus Willd, | 20 | mg |
| root extract at 70% in | | |
| Polysaccharides | | |
| Microcrystalline cellulose | 137 | mg |
| (E460) | | |
| Magnesium salt of fatty acids | 30 | mg |
| (E470b) | | |
| croscarmellose sodium (E468) | 20 | mg |
| talc (E553b) | 10 | mg |
| Silicon oxide | 10 | mg |
| gum arabic (E414) | 3 | mg |
| Amide | 2 | mg |

### Example 5

A further exemplary formulation of the composition according to Invention is described below

| Component | Dose | |
|---|---|---|
| Standardized extract of 85% | 588 mg | |
| bark of Berberis aristata DC | | |
| extract in berberine hydrochloride | | |
| REVIFAST ® | 160 mg (of which 48 mg of | |
| 30% weight resveratrol | resveratrol) | |
| 70% magnesium dihydroxide | | |
| Astragalus microcephalus | 20 | mg |
| Willd, root extract tit at 70% | | |
| in Polysaccharides | | |
| Dehydrated extract of the | 10 | mg |
| Cyclanthera pedata (L.) | | |
| (Caigua) | | |
| Microcrystalline cellulose | 137 | mg |
| (E460) | | |
| Magnesium salt of fatty acids | 30 | mg |
| (E470b) | | |
| croscarmellose sodium | 20 | mg |
| (E468) | | |
| talc (E553b) | 10 | mg |
| Silicon oxide | 10 | mg |
| gum arabic (E414) | 3 | mg |
| Amide | 2 | mg |
| E464, E1521, E171, E553B | 20 | mg |
| and E172 | | |

The composition described in Example 2-5 can be contained in a variety of formulations such as: capsules, tablets, syrups, suspensions and emulsions. The formulations of Examples 2-5 can be administered by splitting them several times a day, preferably twice a day.

### Example 6

An exemplary formulation of the composition according to Invention is described below

| Component | Dose | |
|---|---|---|
| Standardized extract of bark of | 588 mg (of which 500 mg of | |
| Berberis aristata DC extract tit | berberine hydrochloride) | |
| at 85% in berberine hydrochloride | | |
| REVIFAST ® | 160 mg (of which 48 mg of | |
| 30% weight resveratrol | resveratrol) | |
| 70% magnesium dihydroxide | | |
| Red rice extract tit at 5% of | 50 mg (of which 2.5 mg of | |
| monacolin K | monacolin K) | |
| Astragalus microcephalus | 20 | mg |
| Willd, root extract tit at 70% | | |
| in Polysaccharides | | |
| Cross-linked carboxymethyl | 20 | mg |
| cellulose sodium | | |
| Microcrystalline cellulose | 192 | mg |
| (E460) | | |
| Magnesium salt of fatty acids | 30 | mg |
| (E470b) | | |
| croscarmellose sodium (E468) | 20 | mg |
| talc (E553b) | 10 | mg |
| Silicon oxide | 10 | mg |
| gum arabic (E414) | 3 | mg |
| Amide | 2 | mg |

### Example 7

An exemplary formulation of the composition according to Invention is described below

| Component | Dose | |
|---|---|---|
| Standardized extract of bark of | 294 mg (of which 250 mg of | |
| Berberis aristata DC extract tit | berberine hydrochloride) | |
| at 85% in berberine hydrochloride | | |
| REVIFAST ® | 160 mg (of which 48 mg of | |
| 30% weight resveratrol | resveratrol) | |
| 70% magnesium dihydroxide | | |
| Red rice extract tit at 5% of | 50 mg (of which 2.5 mg of | |
| monacolin K | monacolin K) | |
| Dehydrated extract of the | 20 | mg |
| Cyclanthera pedata (L.) | | |
| (Caigua) | | |
| Cross-linked carboxymethyl | 20 | mg |
| cellulose sodium | | |
| Microcrystalline cellulose | 192 | mg |
| (E460) | | |
| Magnesium salt of fatty acids | 30 | mg |
| (E470b) | | |
| croscarmellose sodium (E468) | 20 | mg |
| talc (E553b) | 10 | mg |
| Silicon oxide | 10 | mg |
| gum arabic (E414) | 3 | mg |
| Amide | 2 | mg |

### Example 8

An exemplary formulation of the composition according to Invention and formulated as stick pack is described below

| Component | Dose | |
|---|---|---|
| Standardized extract of bark of | 294 mg (of which 250 mg of | |
| Berberis aristata DC extract tit | berberine hydrochloride) | |
| at 85% in berberine hydrochloride | | |
| REVIFAST ® | 160 mg (of which 48 mg of | |
| 30% weight resveratrol | resveratrol) | |
| 70% magnesium dihydroxide | | |
| Plerotus (Pleurotus ostreatus | 3000 | mg |
| (Jacq.) P. Ku) fungal fruiting | | |
| body e.s. tit. 30% in | | |
| polysaccharides | | |
| Sorbitole | 600 | mg |
| Sucralose | 3 | mg |
| acesulfame K | 1.5 | mg |
| maltodextrin | 200.5 | mg |
| Magnesium stearate | 15 | mg |
| Silicon oxide | 30 | mg |

### Example 9. Case Report on Improving the Lipidic Profile with a Composition According to the Invention Containing Berberine and Resveratrol at Gastric Absorption

Mr. FG is 40 years old and is classified borderline for the lipid profile with an LDL cholesterol level of 3.91 mM. In his dietary supplement scheme, he took a berberine 500 mg supplement separately for 1 month, taking lipoprotein levels to 3.4 mM. Wanting to further lower this lipid level and not wanting to increase the amount of berberine, FG also took a supplement based on resveratrol 50 mg, but did not notice any change compared to the application of berberine alone with regard to the lipid picture. Surprisingly combining the integration of berberine with a resveratrol supported on magnesium hydroxide (Revifast), the lipid values are further improved, reaching the plasma concentration of 2.7 mM. The quantities assumed by Mr. FG are such that the dosage used is that shown in examples 2-5.

Information were also collected from case reports of a number of subjects supplemented in their diet substantially as above. The results are summarized in FIG. 2.

As it can be seen, when the subjects are supplemented with berberine the levels of LDL cholesterol were reduced. Surprising only the combination of berberine/revifast but not the combination of berberine/unsupported resveratrol showed synergy in further reducing LDL cholesterol. It is therefore believed that it is the way in which resveratrol is absorbed that influences the synergistic action of resveratrol to reduce LDL cholesterol by berberine.

1. A pharmaceutical or nutraceutical composition or a food supplement comprising
a mixture of berberine and resveratrol for treatment and/or control of dyslipidemia,
wherein the resveratrol is co-precipitated or supported on a salt or hydroxide of a divalent or trivalent metal.

2. The composition according to claim 1, wherein the resveratrol is supported on magnesium hydroxide.

3. The composition according to claim 1, wherein the berberine is obtained from an extract of Berberis aristata having a berberine content between 50% and 100%.

4. The composition according to claim 1, wherein a ratio by weight between resveratrol and berberine ranges from 0.01 to 1.

5. The composition according to claim 1, further comprising at least one nutrient with properties regulating the lipid profile selected from the group consisting of Astragalus microcephalus, fermented red rice, garlic and catechins from green tea, phytocomplexes derived from Acacia catechu, Acacia farnesiana, Acacia laeta, Acacia vera, Acacia Senegal, Acacia seyal delile, Amaranthus caudatus, Amaranthus cruentus, Amaranthus hypocondria, Cassia mimosoides var. Nomame makino, Cyclantera pedata Schard (Caigua), Ipomea batatas Lamb., Trigonella foenumgraecum and their combinations.

6. The composition according to claim 1, further comprising at least one of
Astragalus microcephalus in an amount between 10 mg and 500 mg on a total weight of the composition between 0.5 g and 2 g,
fermented red rice in an amount between 50 mg and 500 mg on a total weight of the composition ranging from 0.5 g to 2 g,
monocolin k in an amount ranging from 1 mg to 20 mg on a total weight of the composition ranging from 0.5 g to 2 g,
allicin in an amount between 1 mg and 100 mg on a total weight of the composition between 0.5 g and 2 g,
catechins in an amount between 50 mg and 500 mg on a total weight of the composition ranging from 0.5 g to 2 g, and
β-glucan polysaccharides in an amount between 50 mg and 10 g, on a total weight of composition between 0.5 g and 10 g.

7. The composition according to claim 1, further comprising a derivative of a phytocomplex from one or more of Acacia catechu, Acacia farnesiana, Acacia laeta, Acacia vera, Acacia Senegal, Acacia seyal delile, Amaranthus caudatus, Amaranthus cruentus, Amaranthus hypocondria, Cassia mimosoides var. Nomame makino, Cyclantera pedata Schard (Caigua), Ipomea batatas Lamb., Trigonella foenumgraecum in an amount of between 0 mg and 500 mg on a total weight of the composition comprised between 0.5 g and 2 g.

8. A method for treatment and/or control of dyslipidemia in a subject, the method comprising
administering to the subject the composition according to claim 1.

9. The method of claim 8, wherein the composition is administered fractionated daily.

10. The method of claim 8, wherein the administering is performed two times or one time a day.

11. The method of claim 8, wherein the administering is performed for a treatment period of 3 months.

12. The method of claim 8, wherein the administering is performed in association with a statin-based pharmacological therapy.

13. The composition according to claim 1, wherein the ratio by weight between resveratrol and berberine ranges from 0.05 to 0.5.

14. The composition according to claim 3, wherein the extract of Berberis aristata has a berberine content between 70% and 90%.

15. The composition according to claim 6, wherein
the Astragalus microcephalus is in an amount between 15 mg and 100 mg on a total weight of the composition between from 0.75 g to 1.5 g;
the fermented red rice is in an amount from 100 mg to 300 mg on a total weight of the composition ranging from 0.75 g to 1.5 g;
the monocolin k is in an amount ranging from 2 mg to 10 mg, on a total weight of the composition ranging from 0.75 g to 1.5 g;
the allicin is in an amount from 3 mg to 30 mg on a total weight of the composition between 0.75 g and 1.5 g;
the catechins are in an amount from 100 mg to 300 mg even on a total weight of the composition ranging from 0.75 g to 1.5 g; and/or
the β-glucan polysaccharides are in an amount between 500 mg and 5 g on a total weight of composition from 0.75 g to 5 g.

16. The composition according to claim 7, wherein the derivative of the phytocomplex is in an amount from 5 mg to 300 mg on a total weight of the composition between 0.75 g and 1.5 g.
